(19) United States
(12) Patent Application Publication
(10) Pub. No.: US 2017/0214268 A1
(43) Pub. Date: Jul. 27, 2017

(54) Physically-Configurable External Charger for an Implantable Medical Device with Separable Coil and Electronics Housings

(71) Applicant: Boston Scientific Neuromodulation Corporation, Valencia, CA (US)

(72) Inventor: Joshua D. Howard, Winnetka, CA (US)

(21) Appl. No.: 15/354,392

(22) Filed: Nov. 17, 2016

Related U.S. Application Data
(60) Provisional application No. 62/286,253, filed on Jan. 22, 2016.

(51) Int. Cl. H02J 7/02; H02J 50/90; A61N 1/378; H02J 50/10

(52) U.S. Cl. CPC H02J 7/025; H02J 50/10; H02J 50/90; A61N 1/3787

(57) ABSTRACT

A physically-configurable external charger device for an implantable medical device is disclosed, which facilitates the generation of different powers of a magnetic field but with reduced heating concerns at higher powers. The charger includes an electronics housing having control circuitry and a battery, and a coil housing having a charging coil. A cable connects these two housings. The two housings can be connected in a first physical configuration, and separated in a second physical configuration. In the first physical configuration, a low-power magnetic field can be produced, as the electronics housing is connected to the coil housing, and thus may heat to some degree. In a second physical configuration, the electronics housing is removed and extended from the coil housing, and thus a higher-power magnetic field can be produced with reduced heating concerns. Thus, in this second configuration, the charging rate of the IMD can be increased.

### CROSS REFERENCE TO RELATED APPLICATIONS

This is a non-provisional of U.S. Provisional Patent Application Ser. No. 62/286,253, filed Jan. 22, 2016, to which priority is claimed, and which is incorporated herein by reference in its entirety.

This application is also related to U.S. Provisional Patent Application Ser. No. 62/286,257, filed Jan. 22, 2016.

### FIELD OF THE INVENTION

The present invention relates to a wireless charger for an implantable medical device such as an implantable pulse generator.

### BACKGROUND

Implantable stimulation devices are devices that generate and deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder subluxation, etc. The description that follows will generally focus on the use of the invention within a Spinal Cord Stimulation (SCS) system, such as that disclosed in U.S. Pat. No. 6,516,227. However, the present invention may find applicability in any implantable medical device system.

As shown in FIGS. 1A and 1B, a SCS system typically includes an Implantable Pulse Generator (IPG) 10, referred to more generically as an Implantable Medical Device (IMD) 10. IMD 10 includes a biocompatible device case 12 formed of a metallic material such as titanium for example. The case 12 typically holds the circuitry and battery 14 necessary for the IMD 10 to function, although IMDs can also be powered via external RF energy and without a battery, as described further below. The IMD 10 is coupled to electrodes 16 via one or more electrode leads (two such leads 18 are shown), such that the electrodes 16 form an electrode array 20. The electrodes 16 are carried on a flexible body 22, which also houses the individual signal wires 24 coupled to each electrode. In the illustrated embodiment, there are eight electrodes on each lead, although the number of leads and electrodes is application specific and therefore can vary. The leads 18 couple to the IMD 10 using lead connectors 26, which are fixed in a header 28 comprising epoxy for example, which header is affixed to the case 12. In a SCS application, distal ends of electrode leads 18 with the electrodes 16 are typically implanted on the right and left side of the dura within the patient's spinal cord. The proximal ends of leads 18 are then tunneled through the patient's tissue to a distant location such as the buttocks where the IMD 10 is implanted, where the proximal leads ends are then connected to the lead connectors 26.

As shown in cross section in FIG. 2B, the IMD 10 typically includes a printed circuit board (PCB) 30 containing various electronic components 32 necessary for operation of the IMD 10. Two coils are present in the IMD 10 as illustrated: a telemetry coil 34 used to transmit/receive data to/from an external controller (not shown); and a charging coil 36 for receiving power from an external charger 40 (FIG. 2A). These coils 34 and 36 are also shown in the perspective view of the IMD 10 in FIG. 1B, which omits the case 12 for easier viewing. Although shown as inside in the case 12 in the Figures, the telemetry coil 34 can alternatively be fixed in header 28. Coils 34 and 36 may alternative be combined into a single telemetry/charging coil.

FIG. 2A shows a plan view of the external charger 40, and FIG. 2B shows it in cross section and in relation to the IMD 10 as it provides power—either continuously if the IMD 10 lacks a battery 14, or intermittently if the charger is used during particular charging sessions to recharge the battery. In the depicted example, external charger 40 includes two PCBs 42a and 42b; various electronic components 44 for implementing charging functionality; a charging coil 46; and a battery 48 for providing operational power for the external charger 40 and for the production of a magnetic field 60 from the charging coil 46. These components are typically housed within a housing 50, which may be made of hard plastic such as polycarbonate for example.

The external charger 40 has a user interface 54, which typically comprises an on/off switch 56 to activate the production of the magnetic field 60; an LED 58 to indicate the status of the on/off switch 56 and possibly also the status of the battery 48; and a speaker (not shown). The speaker emits a “beep” for example if the external charger 40 detects that its charging coil 46 is not in good alignment with the charging coil 36 in the IMD 10. More complicated user interfaces 54 can be used as well, such as those involving displays or touch screens, or involving realistic audio output (e.g., speech or music) beyond a mere beep, etc.

The external charger's housing 50 is sized such that the external charger 40 is hand-holdable and portable. In an SCS application in which the IMD 10 is implanted behind the patient, the external charger 40 may be placed in a pouch (not shown) around a patient's waist to position the external charger in alignment with the IMD 10. Typically, the external charger 40 is touching the patient's tissue 70 as shown (FIG. 2B), although the patient's clothing or the material of the pouch may intervene.

Wireless power transfer from the external charger 40 to the IMD 10 occurs by near-field magnetic inductive coupling between coils 46 and 36. When the external charger 40 is activated (e.g., on/off switch 56 is pressed), charging coil 46 is driven with an AC current to create the magnetic field 60. The frequency of the magnetic field 60 may be on the order of 80 kHz for example, and may generally be set by the inductance of the coil 46 and the capacitance of a tuning capacitor (not shown) in the external charger 40. The magnetic field 60 transcutaneously induces an alternating current in the IMD 10′s charging coil 36, which current is rectified to DC levels and used to power circuitry in the IMD 10 directly and/or to recharge the battery 14 if present.

The IMD 10 can communicate relevant data back to the external charger 40, such as the capacity of the battery using Load Shift Keying, as explained for example in U.S. Patent Application Publication 2015/0077050, or by any other means. For example, either or both of the charging coil 36 or the telemetry coil 34 can be used to transmit data, or other separate data antennas (e.g., short-range far-field RF antennas, communicating by Bluetooth, WiFi, Zigbee, MICS, or other protocols) can be used in either or both of the IMD 10 and the external charger 40.

Referring again to FIG. 2B, the depicted example of the external charger 40 includes two PCBs 42a and 42b, which are generally orthogonal. The bulk of the electronic components 44 are carried on the vertical PCB 42b. Horizontal PCB 42a by contrast is generally free of components, and carries only the charging coil 46. Further, the battery 48 is placed outside of the area extent of the charging coil 46. As explained in U.S. Pat. No. 9,002,445, such design of the external charger 40 is useful to reduce heating, in particular heating of conductive components resulting from Eddy currents caused by the alternating magnetic field 60. The design moves conductive materials (the PCB 42b with its electronic components 44; the battery 48 with its conductive housing) away from where the magnetic field 60 is most intense in the center of the charging coil 46, as illustrated by the concentration of magnetic field flux lines, shown in dotted lines in FIG. 2C. Further, placing the electronic components 44 on a vertical PCB 42b tends to orient the major planes of the PCB 42b and components 44 parallel to the highest-intensity portions of the magnetic field 60 in the center of the coil 46, rendering such components that much less susceptible to Eddy current heating. The design of the external charger 40 is thus able to remain compact within its hand-holdable housing 50 without significant heating concerns.

Even if heating of the external charger 40 is mitigated by these design choices, it is still prudent to monitor temperature to ensure that a patient will not be injured while charging his IMD 10. In this regard, external charger 40 preferably includes at least one temperature sensor, such as a thermistor 52 (FIG. 2B), to monitor the external charger 40's temperature while charging. Thermistor 52 is preferably placed on the inside surface of the housing 50 that faces (and potentially touches) the patient when the external charger 40 is producing the magnetic field 60.

The thermistor 52 can communicate temperature to control circuitry (part of electronic components 44) within the external charger 70, to ensure that a maximum safe temperature for the patient, Tmax (e.g., 41° C.), is not exceeded. If the thermistor 52 reports this maximum temperature, and particularly in the circumstance where the external charger 40 is used to recharge an IMD 10's battery 14, charging may be suspended by ceasing current through the charging coil 46 to allow the external charger 40 to cool. Once cool enough, for example once the temperature drops to a lower minimum temperature, Tmin (e.g., 39° C.), charging may again be enabled by reinitiating the current through the charging coil 46, until Tmax is again reached and charging suspended, etc. This is illustrated in FIG. 3, and borrowed from U.S. Pat. No. 8,321,029. The patient may not be aware that the external charger 40 is actually duty cycling between enabled and suspended states to maintain a safe temperature during a battery charging session. Other means of temperature control beyond duty cycling exist, such as adjusting the magnitude of the current through the charging coil 46, detuning the frequency of the magnetic field 60, etc.

While external charger 40 works fine to provide power to a patient's IMD 10, the inventor sees room for improvement in external charger design. For example, the inventor notes that while the design of external charger 40 reduces Eddy-current-related heating by moving and orienting components as described above, Eddy current heating will still exist to some degree. As FIG. 2C shows, while the amount of magnetic flux impinging upon the vertically-oriented electronic components 44 and the battery 48 may be lessened, such components are still relatively close to the charging coil 46, and hence still receive magnetic field 60 and will heat to some degree.

The propensity of external charger 40 to heat ultimately impedes its ability to provide significant power to the IMD 10, or to quickly charge the IMD 10's battery 14. This is because Tmax effectively limits the strength of the magnetic field 60 that can be produced, and hence limits the rate at which the battery 14 can be charged.

Accordingly, the inventor proposes a new external charger design that includes separable portions and is also physically configurable. A first physical configuration allows for low-power charging as described to this point, while a second physical configuration allows for high-powered charging, and hence faster IMD battery charging.

### BRIEF DESCRIPTION OF THE DRAWINGS

FIGS. 1A and 1B show an Implantable Medical Device (IMD), in accordance with the prior art.

FIGS. 2A-2C show an external charger for an IMD, in accordance with the prior art.

FIG. 3 shows means for controlling the temperature of the external charger during an IMD battery charging session, in accordance with the prior art.

FIGS. 4A and 4B show an improved external charger in perspective and cross-sectional views respectively, and in a first physical configuration in which an electronics housing is connected to a charging coil housing, in accordance with an example of the invention.

FIG. 5 shows the external charger in a second physical configuration in which the electronics housing is extended from the coil housing by a cable, in accordance with an example of the invention.

FIG. 6 shows plan views of the electronics housing and the coil housing, including various internal structures, in accordance with an example of the invention.

FIG. 7 shows a clasp for connecting the electronics housing and the coil housing, in accordance with an example of the invention.

FIG. 8 shows a cable return for allowing the cable to retract into the electronics housing when the electronics housing and coil housing are connected, in accordance with an example of the invention.

FIGS. 9A and 9B show a means for holding the cable when the electronics housing and coil housing are connected, in accordance with an example of the invention.

FIGS. 10A and 10B show alternative manners of positioning electronics in the electronics housing to reduce Eddy current heating, in accordance with an example of the invention.

FIGS. 11A and 11B show alternative manners in which the electronics housing can be sized and attached to the coil housing, in accordance with examples of the invention.

FIGS. 12A and 12B show use of the external charger to produce low- and high-power magnetic fields for an IMD in conjunction with a charging belt, in accordance with examples of the invention.

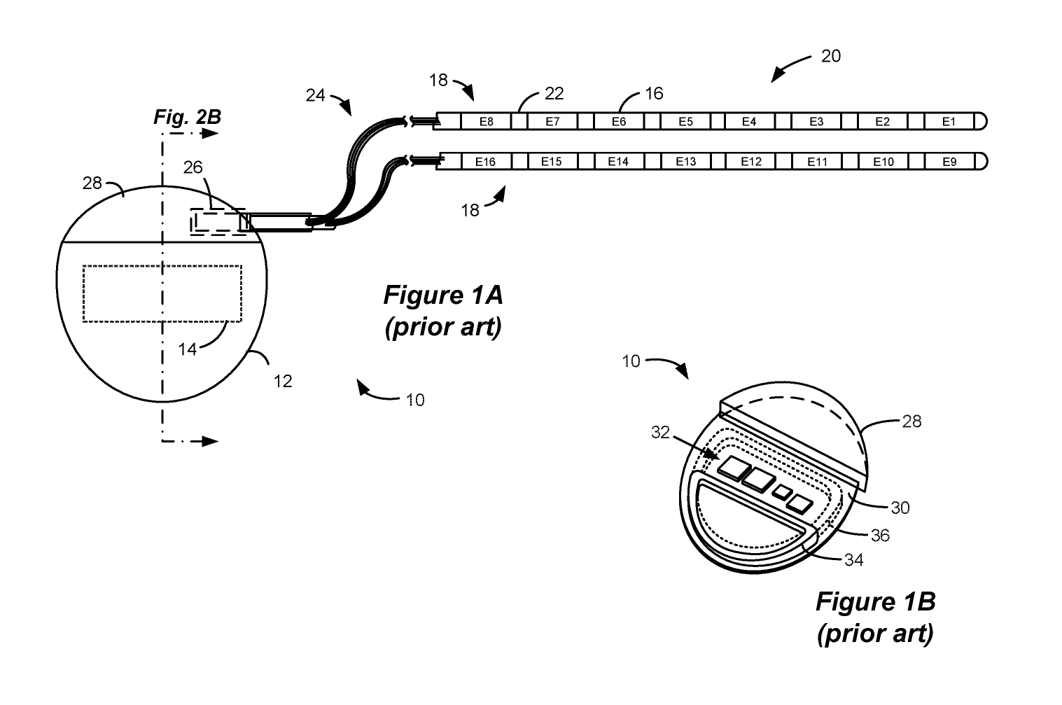

### DETAILED DESCRIPTION

A physically-configurable external charger device for an Implantable Medical Device (IMD) is disclosed, which facilitates the generation of different powers of a magnetic field but with reduced heating concerns at higher powers. The charger includes an electronics housing having control circuitry and a battery, and a coil housing having a charging coil. A cable connects these two housings. The two housings can be connected in a first physical configuration, and separated in a second physical configuration. In the first physical configuration, a relatively low-power magnetic field can be produced, as the electronics housing is connected to and thus near the coil housing, and thus may heat to some degree. In a second physical configuration, the electronics housing is removed and extended from the coil housing preferably by the length of the cable, and thus a higher-power magnetic field can be produced with reduced heating concerns. Thus, in this second configuration, the charging rate of the IMD can be increased.

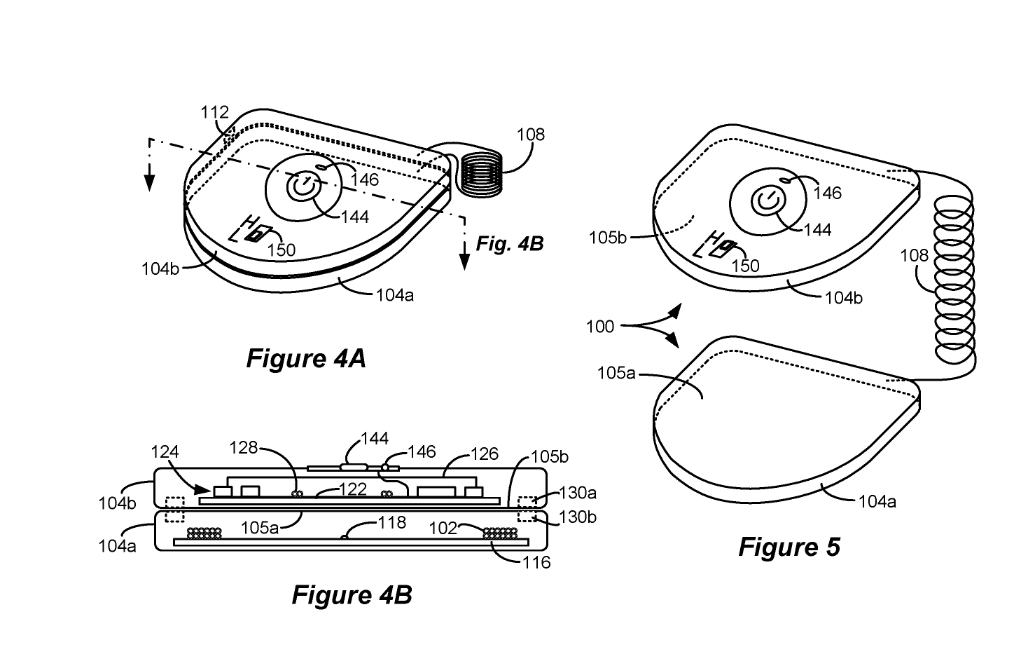

An example of an improved, physically-configurable external charger 100 is shown first in FIGS. 4A and 4B, which respectively show the charger in perspective and cross-sectional views. The external charger 100 includes a housing 104 which as shown comprises two portions, 104a and 104b. Charging coil housing 104a includes a charging coil 102, which like the prior art charger is energized to produce a magnetic field 60 to power and/or charge the IMD 10. Electronics housing 104b includes the majority of the electronics required to operate the external charger 100, including various electronics components 124 (including control circuitry) and a battery 126. With brief reference to FIG. 5, notice that housings 104a and 104b are separable from each other, and connected by a cable 108. This allows the external charger 100 to operate in two different physical configurations—a first (FIG. 4A) in which the housings 104a and 104b are connected, and a second in which the housings 104a and 104b are separated and extended from each other. As explained more fully below, these two different physical configurations facilitate the usage of different power modes in the external charger 100.

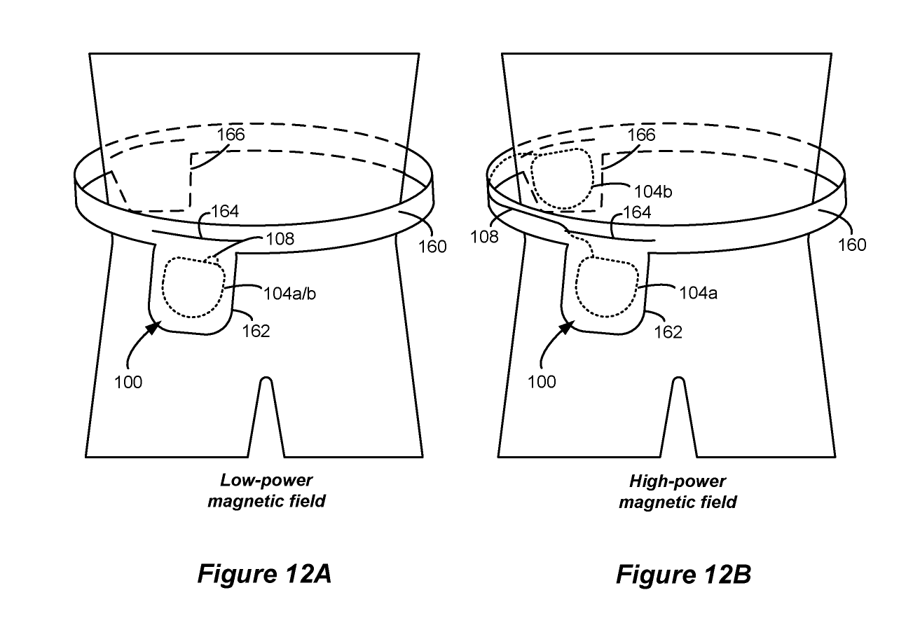

Housings 104a and 104b preferably comprise a hard insulative material such as polycarbonate and have internal cavities to house their respective components. Each housing 104a and 104b may be formed of separate pieces, for example of top and bottom pieces that are bolted together in a “clam shell” arrangement, although this construction detail isn't shown. Note that because the coil housing 104a contains only minimal electronics, as described later, it can be made relatively thin compared to the thickness of the electronics housing 104b. However, as shown in FIG. 4B, the housings 104a and 104b may also be made of the same thickness. The thinness of the coil housing 104a is beneficial because its low profile is less conspicuous when used by a patient to charge his IMD 10, as explained further later with reference to FIGS. 12A and 12B. Housings 104a and 104b can be formed in other ways or of different materials, and some other ways are illustrated subsequently.

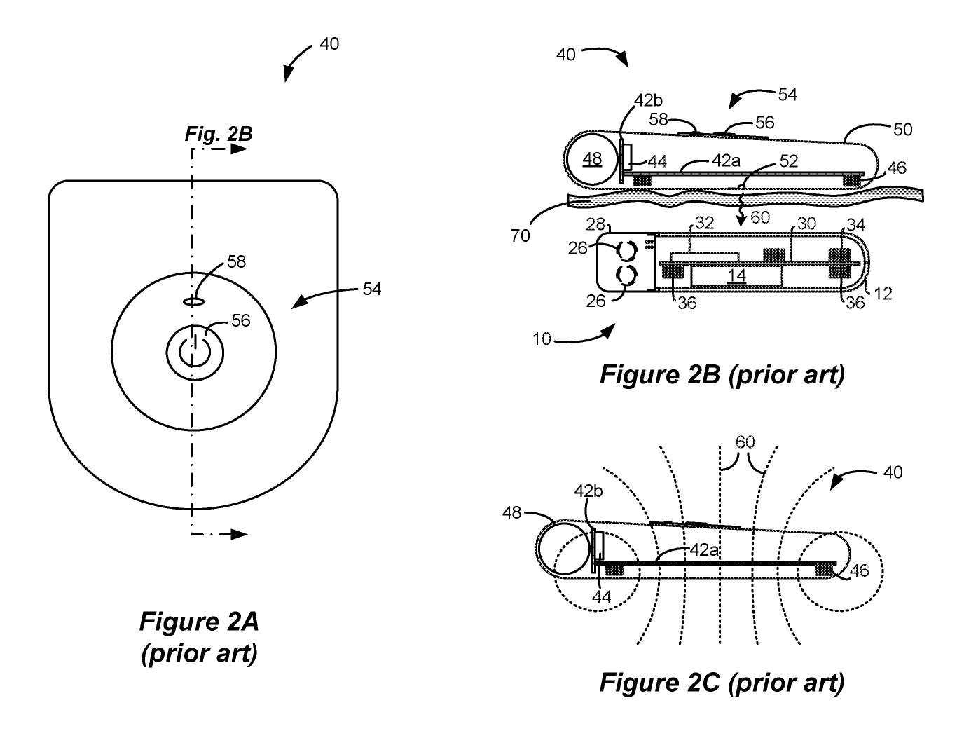

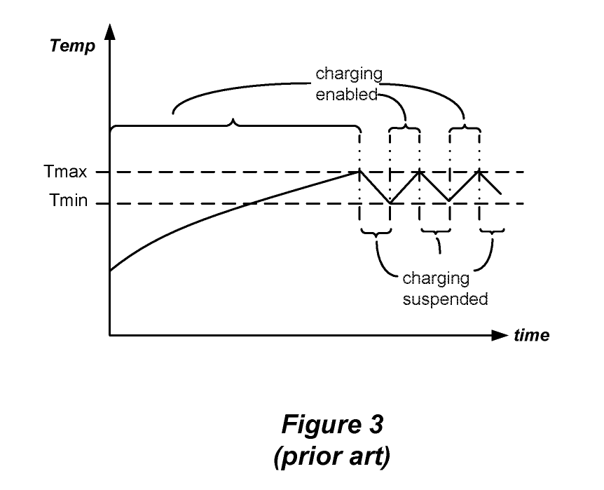

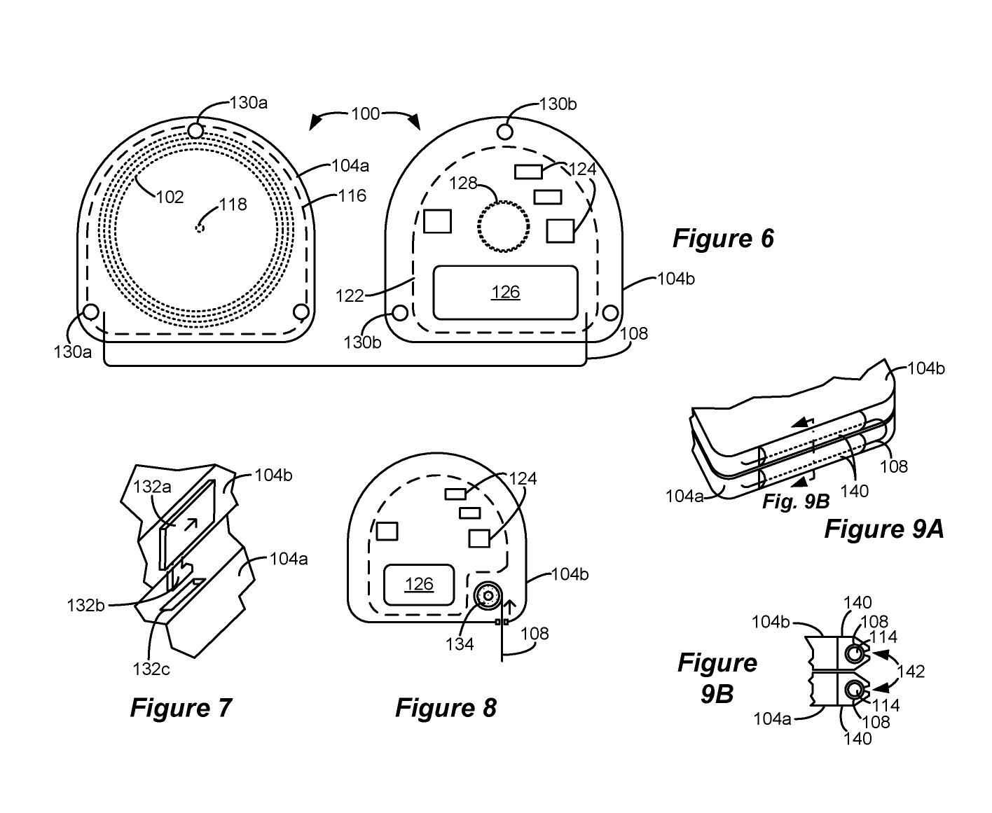

The cross section of FIG. 4B shows the housings 104a and 104b of the external charger 100 as connected and with some of their internal components visible. FIG. 6 also shows these housings 104a and 104b and their components in a plan view. As noted, electronics housing 104b includes electronic components 124 such as control circuitry necessary for charger operation. In this regard, external charger 100 can operate similarly to external charger 40 of the prior art (FIGS. 2A-2C), and electronic components 124 can be generally similar to the electronic components 44 described earlier. Electronics housing 104b also includes a battery 126 as necessary to power the circuitry, and ultimately to provide the power necessary for the charging coil 102 to produce a magnetic field 60. Battery 126 may be either non-rechargeable (primary) or rechargeable (e.g., a Li-ion polymer battery). If battery 126 is rechargeable, it may be recharged via a port 112 (FIG. 4A), and in this regard electronic components 124 within the electronics housing 104b can include battery recharging circuitry, such as is disclosed in U.S. Patent Application Publication 2016/0126771. Port 112 can comprise a mini HDMI port, a mini USB port, and the like, or may be customized.

Electronics housing 104b also preferably includes a user interface, which again can be similar in structure and operation to the user interface of external charger 40; for example, it can include an on/off switch 144 and an LED 146, and possibly also a speaker (not shown). (Power selection switch 150 will be described later). Circuitry in the electronics housing 104b is preferably integrated by a printed circuit board (PCB 122), which also connects to wires 114 (see FIG. 9B) in the cable 108. PCB 122 can be rigid (FR4), or of a flexible type such as Kapton™ Although cable 108 is illustrated as having a hard-wired connection to the electronics housing 104b, it may also connect to the control circuitry in the housing via a connector/port arrangement. For example, one end of cable 108 may couple instead to port 112, which may be positioned anywhere that is convenient on the electronics housing 104b. User interface elements can also appear in different locations on the electronics housing 104b, including elsewhere on its top, on its edges, etc., or can appear on the coil housing 104a.

Coil housing 104a preferably contains only minimal electrical components beyond the charging coil 102. However, as shown, the coil housing 104a may include one or more thermistors 118 (FIGS. 4B and 6) to report temperature to electronic components 124 in the electronics housing 104b. As shown, the thermistor 118 is preferably centered with respect to the charging coil 102. Components within the coil housing 104a can if necessary be supported by a PCB 116, which again can be rigid or flexible. Coil housing 104a may include other circuitry as well, such as driver circuitry for the charging coil 102. Thus, while cable 108 may be coupled to the charging coil 102 via such other circuitry or connections, cable 108 is not necessarily connected directly to the charging coil 102. Cable 108 can again be hard-wired to the coil housing 104a or coupled via a connector/port arrangement.

Having cable 108 connect to the electronics housing 104b and/or the coil housing 104a by a separable connector/port arrangement can be beneficial as it allows one of the housings to be replaced, for example, if either housing 104a or 104b is malfunctioning, or if more advanced technology is developed for either. That being said, permanent hardwired connection of the housings 104a and 104b can also be beneficial as it maintains the external charger 100 ready for use in either physical configuration, as discussed further below. Cable 108 (and any associated connectors/ports) should include enough inner wires 114 (FIG. 9B) to allow for communication between control circuitry in the electronics housing 104b and components in the coil housing 102.

In the example shown in FIGS. 4A and 5, cable 108 is coiled so that it is retracted and takes up a small volume when the electronics housing 104b and the coil housing 104a are connected, as shown in FIG. 4A. When the housings 104a and 104b are separated, the cable 108 will stretch, thus allowing the electronics housing 104b to be separated at a significant distance (e.g., at least six inches) from the coil housing 104a, as shown in FIG. 5. Allowing for separation of the housings moves electronics housing 104b away from the effect of the magnetic field 60 produced by the coil housing 104a, as it either continuously powers the IMD 10, or charges its battery 14 during a charging session. This prevents heating, because conductive structures in the electronics housing 104b—e.g., the PCB 122, electronic components 124, and battery 126—will not be significantly susceptible to Eddy currents caused by the magnetic field 60.

Cable 108 however may be configured differently. For example, cable 108 need not be coiled, and instead could be straight. Because a straight cable 108 might have extra slack, particularly when the electronics housing 104b and coil housing 104a are joined (FIG. 4A), steps can be taken to hold the cable 108 in place. For example, FIG. 9A shows the inclusion of a cable-holding mechanism 140 to retain the cable 108 against the edges of either or both of the electronics housing 104b and coil housing 104a. FIG. 9A shows an example in which cable-holding mechanism 140 comprises a deformable rubberized material including a groove 142 (FIG. 9B) into which the cable 108 can be press fit when the electronics housing 104b and the coil housing 104a are connected (FIG. 4A), and from which the cable 108 can be “peeled” when the two housings are separated (FIG. 5). In the example shown, both the electronics housing 104b and the coil housing 104a have a cable-holding mechanism 140, and so the cable 108 makes a U-turn as it proceeds from one to the other.

Although cable-holding mechanism 140 is shown in FIGS. 9A and 9B as comprising a material separate from the housings 104a and 104b, in other examples it could simply comprise the edges of the housings 104a and 104b as they are formed. Also, cable-holding mechanism 140 could comprise other well-known structures such as clips, clasps, Velcro™, etc. Although not shown, cable-holding mechanism 140 could also comprise a recess formed into either or both of the housings 104a and 104b into which the cable 108 can be stuffed when the housings are connected. Although not shown, cable 108 can also include a stiffening member throughout its length, such as a bendable metal material that allows the cable to retain its shape when bent. This would allow the housings 104a and 104b when separated (FIG. 5) to independently retain their positions with respect to each other.

In another example, the cable 108 can be automatically wound inside of one of the housings 104a or 104b when the electronics housing 104b and coil housing 104a are connected (FIG. 4A) to take up additional slack of the cable 108. This is shown in FIG. 8, which includes a spring-biased cable return 134 which will tend to retract the cable 108 by spiraling the cable 108 around the cable return 134. As one skilled will recognize, such a cable return 134 may have a locking means to prohibit the cable 108 from being retracted when the electronics housing 104b and coil housing 104a are separated. For example, the cable 108 may be pulled outward to allow enough length to separate the housings 104a and 104b, with the cable return 134 locking that length. When desired to reconnect the two housings 104a and 104b, a gentle pull on the cable 108 can release the lock and allow the cable 108 to again be retracted by the cable return 134.

As one skilled in the art will realize, the electronics housing 104b and the coil housing 102 can be securely connected (FIG. 4A) and separable (FIG. 5) in different ways. For example, and as shown in FIGS. 4B and 6, housing 104a can include at least one magnet 130a, and housing 104b can also include at least one magnet 130b. As shown best in FIG. 6, three such magnets 130a may be used in the coil housing 104a, and three magnets 130b may be used in electronics housing 104b and placed in locations corresponding to magnets 130a. As shown in FIG. 4B, the magnets 130a and 130b can be placed on the flat surfaces 105a and 105b of the housings 104a and 104b that mate with each other when the housings are connected. As shown, these magnets 130a and 130b are on the inside of these surfaces 105a and 105b, but could be placed on the outsides as well. Preferably the force of the magnets 130a and 130b is strong enough to hold the housings 104a and 104b together so that the external charger 100 can be used in the first, low-power physical configuration without separating (FIG. 4A), but easy enough to separate by hand when using the external charger 100 in the second, high-power physical configuration (FIG. 5). Different numbers of magnets may be used. Further, magnet(s) may alternatively only be used in one of the housings 104a or 104b, so long as an opposing ferromagnetic material appears in the other housing to provide an attractive force.

The housings 104a and 104b can be connectable and separable in other ways. For example, FIG. 7 shows use of a clasp 132. Clasp 132 comprises a slider 132a coupled to a foot 132b built into an edge of the electronics housing 104b, and further comprises a slot 132c in the coil housing 104a. The slider 132a and foot 132b are spring biased in the direction of the arrow to hold the foot 132b in the slot 132c when the housings 104a and 104b are connected (FIG. 4A). When it is desired to separate the housings 104a and 104b (FIG. 5), a user may slide the slider 132a to oppose the spring bias, allowing the foot 132b to be freed from the slot 132c. One skilled will understand that the housings 104a and 104b may include more than one clasp 132 around its edges. These are just examples, and the housings 104a and 104b can be connectable and separable in other ways, such as by clips, grooves, Velcro™, etc.

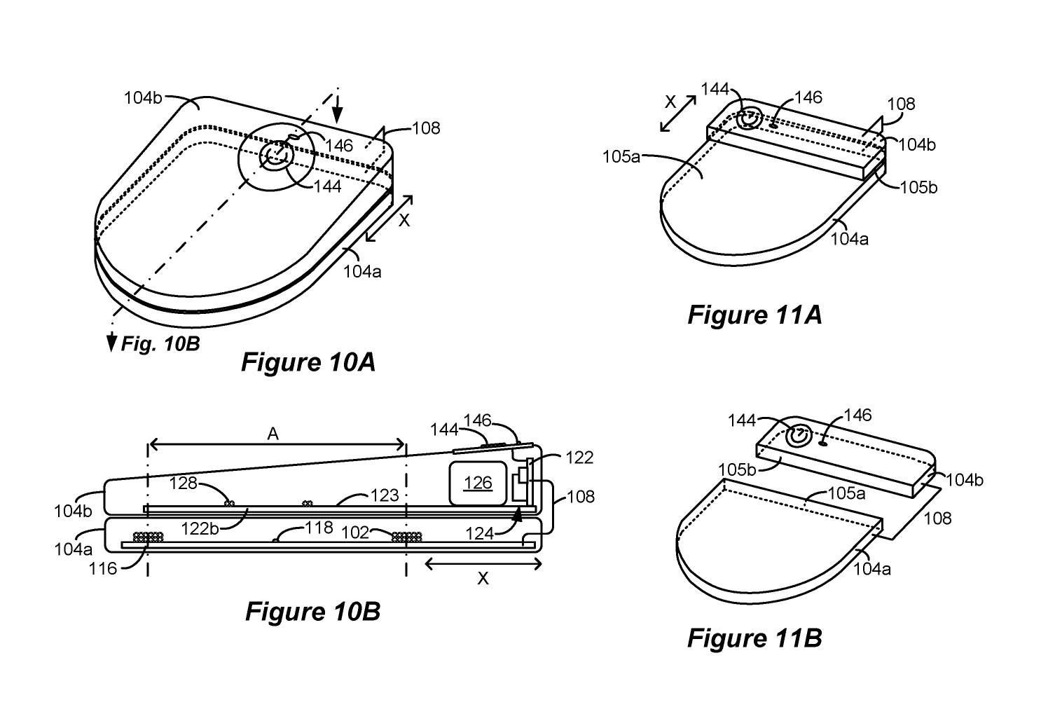

As noted, the external charger 100 is advantageous as regards heating, in that the electronics housing 104b can be moved away from the magnetic field 60 produced by the charging coil 102 in the coil housing 104a. However, the external charger 100 is preferably still operable when the housings 104a and 104b are connected (FIG. 4). In this regard, it can be advantageous to move conductive structures in the electronics housing 104b—more particularly battery 126, PCB 122, and electronic components 124—outside of the area extent of the charging coil 102 even if the housings 104a and 104b are connected. Such a design is shown in one example in FIGS. 10A and 10B and has similarities to the prior art external charger 40 described in the Background. In this example, both housings 104a and 104b are extended by a length X which is outside of the area extent A of the charging coil 102. The mentioned conductive structures in the electronics housing 104b are generally located within the length X to remove them from area A, and thus reduce Eddy current heating in these structures. As discussed in the Background, it can also be advantageous to orient the major planes of the electronics, including the plane of the PCB 122 and the planes of electronic components 124, parallel to highest-intensity portions of the magnetic field 60 present in the center of the charging coil 102, that is, perpendicular to the plane of the coil 102, as shown in FIG. 10B. Notice also that user interface elements, including on/off switch 144 and LED 146, can also be removed from the coil 102's area A.

The electronics housing 104b of FIGS. 10A and 10B is not flat but instead has an angled shape such that the housing 104b is thicker where the battery 126, PCB 122, and electronic components 124 are located. This can be useful to provide more height to accompany the vertically-oriented PCB 122, and possibly the battery 126 as well. However, angling the electronics housing 104b is not strictly required if such structures can be made small enough.

Referring again to FIGS. 4A, 4B and 5, the electronics housing 104b and the coil housing 104a have opposing mating surfaces 105a and 105b that have the same area, and that when connected are parallel to the plane of the charging coil 102, as well as to major planes of the electronics housing 104b and the coil housing 104a. However, this is not necessary, and FIGS. 11A and 11B show other alternatives. In FIG. 11A for example, the opposing surfaces 105a and 105b are not the same area. Instead, surface 105b of the electronics housing 104b is smaller. Further, and preferably, the electronics housing 104b and surface 105b are located in length X that is outside of the area of the charging coil 102, as explained previously with reference to FIGS. 10A and 10B. Electronics housing in FIG. 11A may also be angled or thicker, and its PCB 122 and electronic components 124 oriented vertically, i.e., perpendicular to the plane of the charging coil 102, as also previously discussed.

FIG. 11B provides another alternative in which the opposing surfaces 105a and 105b are located on the edges of the housings 104a and 104b and are perpendicular to the plane of the charging coil 102 when the housings 104a and 104b are connected. In this example, the cable 108 may connect to the edges of the electronics 104b and coil 104a housings to allow the surfaces 105a and 105b to mate without interference. However, cable 108 may also connect to the top or bottom surfaces of the housings 104a and 104b as well. Electronics housing 104b in FIG. 11B may again be angled or have vertically-oriented components as previously described.

With the structure of the external charger 100 explained, attention now turns to use of the external charger 100, and particularly use of the external charger in different power modes. An advantage to the design of external charger 100 is that its physical configurability—in which electronics housing 104b can either be connected to (FIG. 4A) or removed from (FIG. 5) the coil housing 104a—facilitates different power levels to be used to produce the magnetic field 60 for the IMD 10.

Specifically, the first configuration of FIG. 4A in which the electronics housing 104b is connected to the coil housing 104a allows for the external charger 100, specifically control circuitry in electronics housing 104b, to energize the charging coil 102 to produce a magnetic field 60 of a normal power level, comparable to the external charger 40 of the prior art. Such a normal power level is referred to as “low” for comparative purposes. By contrast, the second configuration of FIG. 5 in which the electronics housing 104b is removed and extended from the coil housing 104a allows the external charger 100 to similarly produce a higher-power magnetic field 60. This is because the extended configuration moves the majority of conductive structures of the external charger 100—including significantly the battery 126, PCB 122, and components 124—significantly far away from the influence of the magnetic field 60 that Eddy current heating is mitigated. Magnetic field 60 may thus be of higher power while at the same time being less likely to exceed a safe operating temperature (Tmax) for the external charger 100. This is beneficial to the IMD powering process as a whole, because the IMD 10 can receive and use higher amounts of power (should it lack a battery 14), and/or because the battery 14 in the IMD 10 can be charged at a faster rate.

The electronic components 124 in the electronics housing 104b, in particular its control circuitry, can produce a low- or high-power magnetic field 60 in a number of ways. For example, a low-power magnetic field can be produced by passing a relatively low AC current through the charging coil 102, while a high-power magnetic field can be produced by passing a higher AC current. In another approach, a low-power magnetic field can be produced by passing an AC current through the charging coil 102 with a relatively low duty cycle—i.e., a low on-to-off ratio. A high-power magnetic field by contrast may use the same magnitude of the coil current, but may increase the duty cycle.

The electronics housing 104b is operable to produce a low- or high-power magnetic field 60 in different manners. One way, shown in FIGS. 4A and 5, is to include a control mechanism as part of the user interface of the external charger 100 to allow the user to choose a low- or high-power magnetic field 60. Specifically, a switch 150 is carried by the electronics housing 104b that allows a user the option to select a low-power (“L”) or high-power (“H”) magnetic field 60. Preferably the patient would make these choices with the external charger 100 in the proper physical configuration as described above, although this isn't required.

Alternatively, whether external charger 100 produces a low- or high-power magnetic field 60 can occur automatically depending on the physical configuration of the external charger 100. This requires electronic components 124 in the electronics housing 104b to detect whether the electronics housing 104b is connected to or removed from the coil housing 104a, and such automatic detection and magnetic field generation can occur in different ways. For example, although not shown, either or both of the housings 104a or 104b could include a pressure switch that is engaged when the electronics housing 104b is connected to the coil housing 104a.

In another example, shown in FIGS. 4B and 6, the electronics housing 104b may include a detection coil 128. The inductance of the detection coil 128 can be monitored, with changes in its inductance affected by the physical configuration of the two housings 104a and 104b. When the housings 104b and 104a are connected and thus coils 128 and 102 are relatively close, the inductance of the detection coil 128 will be affected by mutual inductance formed with charging coil 102. By contrast, when the electronics housing 104b is removed and extended from the coil housing 104a, the inductance of the detection coil 128 will remain unaffected by the charging coil 102. If necessary, detection coil 128 can be supported by a horizontal PCB—for example, the PCB 122 of FIGS. 4B and 6, or the additional PCB 123 provided in the example of FIG. 10B. Detection coil 128 may also be formed in the traces of those PCBs. These are merely examples, and other means of automatically detecting the physical configuration of the external charger 100 and automatically adjusting the power of the magnetic field 60 will be recognized by those skilled in the art.

Note that whether the external charger 100 is producing a low- or high-power magnetic field 60, temperature control as described earlier can still be enabled in the external charger 100 as assisted by temperature data provided by the thermistor(s) 118 (FIGS. 4B and 6). Note further that low- and high-power magnetic fields need not be constant power levels. In other words, the control circuitry in the electronics housing 104b may adjust the magnitude of both the low- or high-power magnetic fields 60 depending for example on coupling with the IMD 10, temperature detection, or for other reasons known in the art.

External charger 100 is generally sized similarly to the external charger 40 of the prior art when the housings 104a and 104b are connected, and is hand-holdable and portable. The manner in which external charger 100 is used by a patient is also generally similar, although modified depending on the external charger 100's physical configuration and/or the power level it is producing. FIG. 12A shows external charger 100 when the electronics housing 104b and coil housing 104a are connected, and when used to produce a low-power magnetic field. FIG. 12B shows use when electronics housing 104b is removed and extended from coil housing 104a to produce a high-power magnetic field.

In both examples, a charging belt 160 is used, similar to that described in U.S. Patent Application Publication 2014/0025140. The belt 160 has a pouch 162 which in this example is shown at the back of a patient near to where the IMD 10 (not shown) would be implanted in an SCS application. If a low-power magnetic field is to be used as shown in FIG. 12A, the housing portions 104a and 104b are connected, and the entire external charger 100 is slipped into pouch 162 by an opening 164 in the belt. If a high-power magnetic field is to be used as shown in FIG. 12B, the coil housing 104a with its charging coil 102 (not shown) can remain in the pouch 162, while the electronics housing 104b and cable 108 are removed through opening 164 and extended away from the coil housing 104a. The extended electronics housing 104b as shown in FIG. 12B may be placed into a second pouch 166 on the belt 160, which pouch 166 may be more proximate to the front of the patient, assuming cable 108 is long enough. This beneficially reduces heating in the electronics housing 104b, and further beneficially places user interface aspects of the external charger 100 to where they may be more easily accessed by the patient. However, the extended electronics housing 104b could be placed elsewhere, such as in an opposing pants pocket, etc. It should be understood that while the external charger 100 is shown as operable in conjunction with a belt 160, this is only one example of a usage model, and therefore not the only manner in which the external charger 100 can be used.

Note that the variations and alternatives shown and described for the external charger 100 can be used together in any combination, even if such variations and alternatives are not expressly shown in the Figures or discussed in the text.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. An external charger for an implantable medical device, comprising:
an electronics housing comprising control circuitry and a battery;
a coil housing comprising a charging coil; and
a cable coupled at a first end to the charging coil in the coil housing and connected at a second end to control circuitry in the electronics housing,
wherein the control circuitry is configured to energize the charging coil via the cable to produce a magnetic field to provide power to the implantable medical device, and
wherein the electronics housing and coil housing are configured to be connectable to establish a first configuration for the external charger, and configured to be separable to establish a second configuration for the external charger.

2. The external charger of claim 1, wherein the electronics housing comprises a first flat surface, the coil housing comprises a second flat surface, and wherein the first and second surfaces are mated when the electronics housing and coil housing are connected in the first configuration.

3. The external charger of claim 2, wherein the first and second surfaces are parallel to a major plane of the electronics housing and are parallel to a major plane of the coil housing when the electronics housing and coil housing are connected in the first configuration.

4. The external charger of claim 2, wherein the first and second surfaces are parallel to a plane of the charging coil when the electronics housing and coil housing are connected in the first configuration.

5. The external charger of claim 2, wherein the first and second surfaces are perpendicular to a plane of the charging coil when the electronics housing and coil housing are connected in the first configuration.

6. The external charger of claim 2, wherein the first and second surfaces have the same area.

7. The external charger of claim 2, wherein the first and second surfaces are located at edges of the electronics housing and the coil housing.

8. The external charger of claim 1, wherein the electronics housing and the second housing have the same thickness.

9. The external charger of claim 1, wherein the electronics housing further comprises a circuit board for the control circuitry, and wherein the circuit board is perpendicular to a plane of the coil when the electronics housing and coil housing are connected in the first configuration.

10. The external charger of claim 1, wherein the charging coil has an area, and wherein the control circuitry and the battery are outside of the area when the electronics housing and coil housing are connected in the first configuration.

11. The external charger of claim 1, wherein the electronics housing comprises a port, and wherein the battery is rechargeable via the port.

12. The external charger of claim 1, wherein the cable is coiled.

13. The external charger of claim 1, wherein either or both of the electronics housing or the coil housing is configured to retract the cable into that housing when the electronics housing and coil housing are connected in the first configuration.

14. The external charger of claim 1, wherein either or both of the electronics housing or the coil housing comprises a cable-holding mechanism configured to retain the cable when the electronics housing and coil housing are connected in the first configuration.

15. The external charger of claim 1,
wherein the control circuitry is operable to energize the charging coil to produce the magnetic field of a first power when the electronics housing and coil housing are connected in the first configuration, and
wherein the control circuitry is operable to energize the charging coil to produce the magnetic field of a second power when the electronics housing is separated from the coil housing in the second configuration.

16. The external charger of claim 15, wherein the second power is higher than the first power.

17. The external charger of claim 15, further comprising a user interface, wherein producing the first power or the second power is selectable as an option on the user interface.

18. The external charger of claim 15, wherein the control circuitry is configured to automatically detect whether the electronics housing and coil housing are connected in the first configuration or separated in the second configuration and automatically produces the magnetic field with the first power or the second power respectively.

19. A method for providing power to an implantable medical device using an external charging device, comprising:
using an electronics housing of the external charging device to energize a charging coil within a coil housing of the external charging device to produce a magnetic field of a first power while the electronics housing is connected to the coil housing; and
using the electronics housing to energize the charging coil to produce a magnetic field of a second power while the electronics housing is separated from the coil housing.
